(19) United States
(12) Patent Application Publication
(10) Pub. No.: US 2023/0183641 A1
(43) Pub. Date: Jun. 15, 2023

(54) SCAFFOLD FOR CELL OR TISSUE CULTURE, THE PREPARING METHOD AND USE THEREOF IN TISSUE ENGINEERING AND REGENERATIVE MEDICINE

(71) Applicants: National Health Research Institutes, Zhunan Township, Miaoli County (TW); National Tsing Hua University, Hsinchu (TW)

(72) Inventors: Shaw-Fang YET, Zhunan Township, Miaoli County (TW); Jane WANG, Hsinchu (TW); Wei-Cheng JIANG, Zhunan Township, Miaoli County (TW); Wai-Sam AO-IEONG, Hsinchu (TW); Syuan-Ku HSIAO, Hsinchu (TW)

(21) Appl. No.: 17/922,268

(22) PCT Filed: Apr. 29, 2021
(86) PCT No.: PCT/US21/30000

§ 371 Date: Oct. 28, 2022

Related U.S. Application Data
(60) Provisional application No. 63/017,562, filed on Apr. 29, 2020.

(51) Int. Cl. C12N 5/00

(52) U.S. Cl. CPC C12N 5/0062; C12N 5/0068; C12N 2533/30; C12N 2513/00

(57) ABSTRACT

The present disclosure relates to a scaffold with staircase microstructure for cell or tissue culture, comprising multiple layers. Each layer defines a plurality of through holes, and the through holes of each layer is in communication with a corresponding through holes of an adjacent layer. A method for culturing cell and tissue regeneration is also provided.

### FIELD OF THE DISCLOSURE

The present disclosure relates to a field of cell culture; more particularly, it relates to a scaffold with staircase microstructure for cell or tissue culture, and a method of tissue engineering and regeneration by using the scaffold.

### BACKGROUND OF THE DISCLOSURE

The regeneration of damaged or lost tissue is a critical step toward realizing full organ regeneration in modern medicine. Despite the advent of surgical techniques, repairing malfunctioned tissues remains particularly difficult. In the development of tissue engineering, the transmission of internal substance is affected by the permeability due to the lack of appropriate biological scaffolds or the help of vascularization. It also results in the restriction of the construction of large or complex biological tissues.

In the traditional preparing method of cell culture scaffolds, it is hard to control the fine structure of scaffold, resulting in excessive pores and insufficient surface area for cell attachment. Therefore, with increasing popularity of scaffold applications toward tissue engineering, scaffolds with the design of high cell adhesion and medium diffusion abilities for regeneration of defective tissue are especially in high demand. Although many of natural or polymeric materials are utilized for fabrication of the scaffolds with various shaped frames to mimic porous structures, insufficient surface area for cell seeding and attachment and low medium diffusion remain an open issue.

Poly(glycerol sebacate) (PGS) is a new low-cost elastomeric polymer with biocompatible and biodegradable properties at the same time. Its components, glycerol and sebacic acid, are physiological metabolites in mammals and are approved by the FDA for biomedical applications. However, the traditional producing process of PGS requires a high temperature and low pressure environment, which limits its application in tissue engineering.

Thus, there is a need for developing an improved scaffold for culturing cell in use of tissue engineering and regenerative medicine fields.

### SUMMARY OF THE DISCLOSURE

In one aspect, the present invention provides a scaffold for culturing cell to promote not only cell seeding efficiency and culture medium diffusivity but also tissue integration in the wound after implantation. The scaffold is made of a light-curable and biodegradable polymer, poly(glycerol sebacate) acrylate (PGSA), which is an acrylation- modified PGS and can be produced by light-curing technology to avoid the high temperature and low pressure environment for PGS.

Accordingly, the present disclosure provides a scaffold with staircase microstructure for cell or tissue culture, comprising:
a first layer comprising a plurality of first through holes with a regular polygon shape; and
a second layer comprising a plurality of second through holes with a regular polygon shape, wherein the first layer is stacked on and adjacent to the second layer in staggering order;
wherein the first layer is stacked on and adjacent to the second layer in staggering order, and one of the first through holes is in communication with a corresponding one of the second through holes; and wherein the center of each first through hole is respectively aligning with a vertex of the corresponding second through hole.

In one embodiment, the size of the first through holes is substantially as same as the size of the second through holes.

In one embodiment, the orientation of the first through holes differs from the orientation of the second through holes.

In one embodiment, the regular polygon shape is a triangle, square, pentagon hexagon, preferably hexagon.

In one embodiment, each of the first through holes and the second through holes further comprises a connecting bar.

In one embodiment, the connecting bars are disposed within one of the first through holes and one of the second through holes, and the scaffold for cell culture further comprises a central bar connecting the connecting bars.

In one embodiment, the first through holes and the second through holes are enclosed through holes.

In another preferred embodiment, the sides of the first through holes are discontinuous segments, and each of the first through holes is framed by six segments indirectly connecting with one another.

In one embodiment, each of the connecting bars of the first layer connects with two segments.

In one embodiment, the plurality of segments is arranged to form one of the first through holes, and the segments are not connected with one another.

In one embodiment, a gap is formed between two adjacent segments.

In one embodiment, each of the segments includes at least two portions intersected with each other.

In one embodiment, each of the second through holes is an enclosed through hole, and each of the first through holes is a space surrounded by a plurality of discontinuous segments.

In one embodiment, the scaffold for cell culture and tissue regeneration can further comprises multiple copies of the first layer and the second layer.

In one preferred embodiment, the scaffold for cell culture and tissue regeneration further comprises two more copies of the first layer and the second layer, wherein is a third layer with a plurality of third through holes, a fourth layer with a plurality of fourth through holes, a fifth layer with a plurality of fifth through holes, and a sixth layer with a plurality of fifth through holes, respectively; wherein the third layer and the fifth layer are substantially as same as the first layer and the fourth and the sixth layer are substantially as same as the second layer.

In one embodiment, the third layer is stacked on and adjacent to the fourth layer in staggering order and the third layer is also adjacent to and under the second layer in staggering order, and the fifth layer is stacked on and adjacent to the sixth layer in staggering order and the fifth layer is also adjacent to and under the fourth layer in staggering order to form a scaffold with six layers stacked in a spiral staircase way.

In one embodiment, the sides of the first through holes, the third through holes and the fifth through holes are discontinuous segments, and each of the first through holes, the third through holes and the fifth through holes is framed by six segments indirectly connecting with one another.

In one embodiment, each of the connecting bars of the first layer, the third layer and the fifth layer connect with two segments.

In one embodiment, the plurality of segments is arranged to form the first through holes, the third through holes and the fifth through holes, and the segments are not connected with one another.

In one embodiment, a gap is formed between two adjacent segments.

In one embodiment, each of the segments includes at least two portions intersected with each other.

In one embodiment, each of the second through holes, the fourth through holes and the sixth through holes is an enclosed through hole, and each of the firth through hole, third through holes and the fifth through holes is a space surrounded by a plurality of discontinuous segments.

In one embodiment, the scaffold for cell culture and tissue regeneration is made of a biocompatible material, preferably is poly(glycerol sebacate) acrylate (PGSA).

In other aspect, the present disclosure provides a method for culturing a cell or culturing a vascularization tissue comprising culturing the cell with the scaffold as mentioned above.

In one embodiment, the cell is an embryonic cell or vascular progenitor cell.

The present disclosure provides a method for enhancing differentiation of a stem cell comprising culturing the stem cell with the scaffold as mentioned above.

In one embodiment, the stem cell is an embryonic stem cell.

In other aspect, the present disclosure provides a method for enhancing differentiation of a vascular cell comprising culturing the vascular cell with the scaffold as mentioned above.

In one embodiment, the vascular cell is a vascular progenitor cell.

In another aspect, the present disclosure provides a method for enhancing vascularization in a wound comprising:
culturing a vascular cell with the scaffold as mentioned above for generating a pre-endothelialized engraftment scaffold,
engrafting the pre-endothelialized engraftment scaffold into the wound.

In one embodiment, the vascular cell is a vascular progenitor cell.

In other aspect, the present disclosure provides a method for enhancing engraftment of a cell comprising culturing the cell with the scaffold e as mentioned above.

In one embodiment, the cell is an embryonic cell or vascular progenitor cell.

The present disclosure is described in detail in the following sections. Other characteristics, purposes and advantages of the present disclosure can be found in the detailed description and claims.

### BRIEF DESCRIPTION OF THE DRAWINGS

FIG. 1 shows a top view of a scaffold for cell culture according to one embodiment of the disclosure.

FIG. 2 shows a scanning electron microscopic view of a scaffold for cell culture according to one embodiment of the disclosure.

FIG. 3 shows a scanning electron microscopic view of a scaffold for cell culture according to one embodiment of the disclosure.

FIG. 4 shows a three-dimensional schematic representation of a scaffold for cell culture according to one embodiment of the disclosure.

FIG. 5 shows a scanning electron microscopic view of a scaffold for cell culture according to one embodiment of the disclosure.

FIG. 6 shows a scanning electron microscopic view of a scaffold for cell culture according to one embodiment of the disclosure.

FIG. 7 shows a top view of a layer according to one embodiment of the disclosure.

FIG. 8 shows a three-dimensional schematic representation of a scaffold for cell culture according to one embodiment of the disclosure.

FIG. 9A-9B show spontaneous differentiation of ESCs on different substrates. (A) RT² profiler PCR array analysis of ESCs on gelatin and PGSA after one-week culture without LIF. (B) qRT-PCR analysis of specific germ layer markers one week after spontaneous differentiation of ESCs on gelatin or PGSA. Gene expression of ESCs is set as 1. Values are mean±SE of three experiments. *P<0.05.

FIG. 10A-10D show VPCs on different matrices during endothelial cell differentiation. (A) EC-related gene expressions on fibronectin, collagen IV and PGSA one week after EC differentiation are measured by qRT-PCR. Groups with different letters are significantly different, whereas groups with same letters are not, *P<0.05. Values are mean±SE of three experiments. (B) Morphological changes of VPCs to differentiated ECs on PGSA substrate. (C) Comparison of gene transcription levels after four weeks of EC induction between culturing on collagen IV and PGSA. (D) Comparison of gene transcription levels after two weeks of SMC induction between culturing on collagen IV and PGSA. Data are presented as means±SE from three experiments, *P<0.05.

FIG. 11A-11D show different design of PGSA scaffolds. (A) hexagonal well scaffolds (B) hexagonal staggered scaffolds (C) hexagonal staircase scaffolds (D) high diffusion hexagonal staircase scaffolds.

FIG. 12A-12C show creation of vascular constructs by VPCs and high diffusion staircase PGSA scaffolds. (A) Design of novel six layers of rotating hexagonal and high diffusion staircase scaffold. (B) Macro- and micro-structure of 3D-printed PGSA scaffold. (C) Schematic diagrams of vascular constructs in transwell by suspension culture under four weeks EC induction.

FIG. 13A-13D show in vitro and in vivo test of vascular constructs. (A) Scanning electron micrograph of high diffusion hexagonal staircase PGSA scaffold only and VPC-ECs on scaffold. (B) Histological staining of VPCs at four weeks post EC differentiation and expression of PECAM1 in vascular construct. (C) Laser speckle contrast images of vascular construct after four weeks subcutaneous implanted in mice. (D) Morphology and functionality of vascular constructs after subcutaneous transplantation for four weeks as demonstrated by H&E staining and immunohistological staining for PECAM1.

FIG. 14A-14C show transplantation of vascular constructs in wound healing mice. (A) Representative images of mice back skin after injury and after implantation of control (PBS), scaffold only, VPC-ECs on disc or scaffold at day 10. Laser speckle contrast images of lesion sites in mice after transplantation of PBS, scaffold, VPC-ECs on disc or scaffold for 10 days. (B) Quantification of blood flux is measured by laser speckle contrast images after 10 days transplantation. Data represent mean values ±SEM and the solid black line denotes mean value; P<0.05. (C) PECAM1 immunohistochemistry is performed to identify endothelial cells (brown) on longitudinal sections of wounds from treatment of PBS, scaffold, VPC-ECs on disc or scaffold at day 10. Magnified areas from upper panels, respectively.

### DETAILED DESCRIPTION OF THE DISCLOSURE

The present disclosure can be more readily understood by reference to the following detailed description of various embodiments of the disclosure, the examples, and the chemical drawings and tables with their relevant descriptions. It is to be understood that unless otherwise specifically indicated by the claims, the disclosure is not limited to specific preparation methods, carriers or formulations, or to particular modes of formulating the extract of the disclosure into products or compositions intended for topical, oral or parenteral administration, because as one of ordinary skill in the relevant arts is well aware, such things can, of course, vary. It is also to be understood that the terminology used herein is for the purpose of describing particular embodiments only and is not intended to be limiting.

As utilized in accordance with the present disclosure, the following terms, unless otherwise indicated, shall be understood to have the following meaning:

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint.

It must be noted that, as used in the specification and the appended claims, the singular forms “a,” “an” and “the” include plural referents unless the context clearly dictates otherwise. Thus, unless otherwise required by context, singular terms shall include the plural and plural terms shall include the singular.

As used herein, the term “or” in the claims is used to mean “and/or” unless explicitly indicated to refer to alternatives only or the alternatives are mutually exclusive.

As used herein, the terms “first,” “second,” etc. refer to different units (for example, a first layer, a second layer). The use of these terms herein does not necessarily connote an ordering such as one unit or event occurring or coming before another, but rather provides a mechanism to distinguish between particular units.

### EXAMPLES

### Example 1. The Structure of the Scaffold with Hexagonal Microstructure for Cell Culture and Tissue Regeneration

In order to promote not only cell seeding efficiency and culture medium diffusivity but also tissue integration in the wound after implantation, a scaffold for cell culture with high cell adhesion and medium diffusion abilities for regeneration of defective tissue is provided in the disclosure. According to the disclosure, a scaffold for cell culture and tissue regeneration comprising multiple layers is provided. Each layer defines a plurality of through holes, and the through holes of each layer is in communication with a corresponding through holes of an adjacent layer. Particularly, a scaffold for cell culture and tissue regeneration according to the disclosure comprises:
a first layer comprising a plurality of first through holes with a regular polygon shape; and
a second layer comprising a plurality of second through holes with a regular polygon shape;
wherein the first layer is stacked on and adjacent to the second layer in staggering order, and one of the first through holes is in communication with a corresponding one of the second through holes; and wherein the center of each first through hole is respectively aligning with a vertex of the corresponding second through hole.

The scaffold for cell and tissue culture according to the disclosure provides a substrate for culturing a cell which preferably needs attachment. For enlarging a culture capacity, a ratio of a surface area to a volume is preferably increased. In one aspect, a size of the first through hole or the second through hole is decreased.

The scaffold for cell and tissue culture according to the disclosure comprises a plurality of layers. The layers may be connected to each other or disconnected to each other, preferably connected to each other. Preferably, a layer according to the disclosure is adhered to an adjacent layer.

It is believed, though not intended to be restricted by any theoretical, that since one of the first through holes is in communication with a corresponding one of the second through holes, medium for the cell culture is able to flow between the first layer and the second layer.

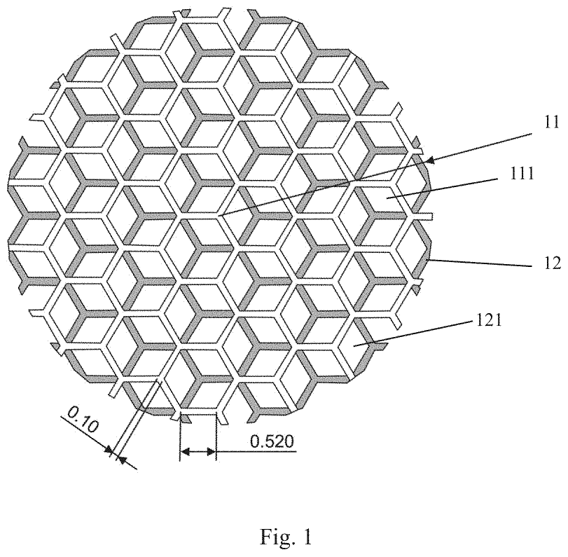

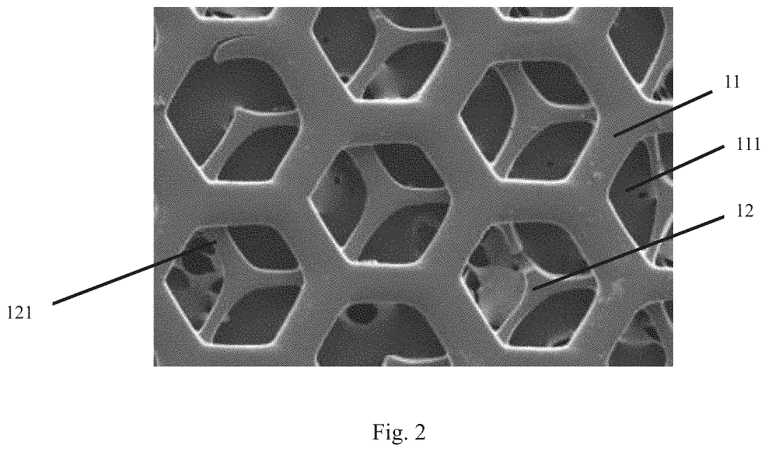

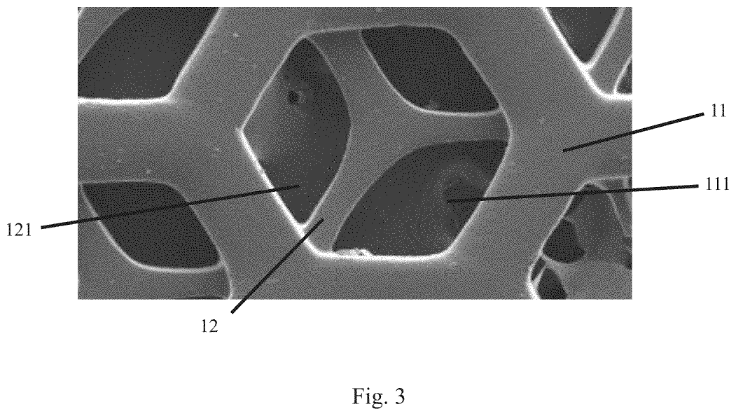

Referring to FIGS. 1 to 3, the scaffold for cell and tissue culture comprises a first layer 11 defining a plurality of first through holes 111; and a second layer 12 defining a plurality of second through holes 121, wherein the first layer 11 is stacked on and adjacent to the second layer 12 in staggering order, and the center of the first through holes 111 is aligning with the vertex of the second through holes 121, wherein one of the first through holes 111 is in communication with a corresponding one of the second through holes 121.

Preferably, a size of the first through holes is substantially same as a size of the second through holes. The first through holes or the second through holes may be in any shapes, such as triangle, square, pentagon, hexagon, preferably hexagon. In another aspect, an orientation of the first through holes differs from an orientation of the second through holes, wherein the orientation of the first through holes is preferably clockwise rotating for 30°, 60°, 90°, 120°, 150°, or 180° relative to the second through holes; preferably 120°.

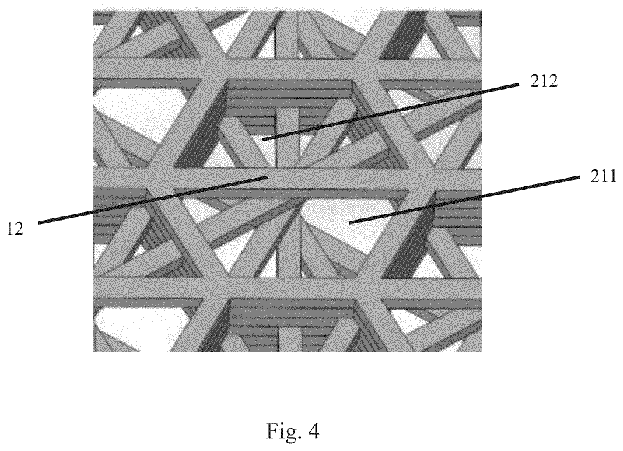

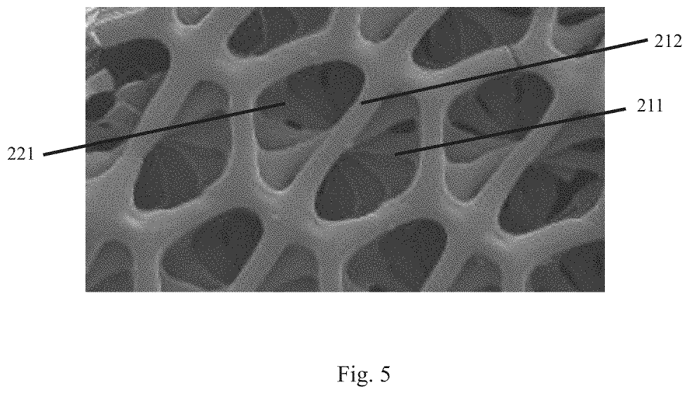

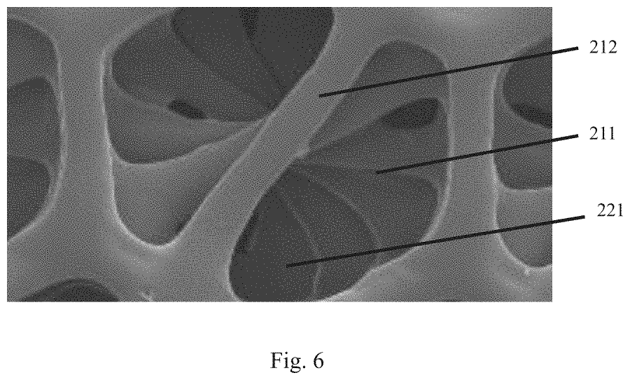

Referring to FIGS. 4 to 6, the scaffold for cell and tissue culture further comprises a connecting bar 212 disposed within one or a plurality of the first through holes 211 or one or a plurality of the second through holes 221. Preferably, the connecting bars are disposed within the entire first through holes and the entire second through holes, and the scaffold further comprises a central bar to connect the connecting bars.

The first through holes or the second through holes may be enclosed through holes or open through holes. Preferably, the first through holes and the second through holes are enclosed through holes. In another aspect, the first through holes are preferably open through holes and the second through holes are preferably enclosed through holes. In another aspect, the first through holes are preferably enclosed through holes and the second through holes are preferably open through holes. It is believed, though not intended to be restricted by any theoretical, that the open through holes benefits medium for the cell culture to flow between the through holes of the same layer.

### Example 2. The Structure of the Scaffold with Hexagonal Staircase Microstructure for Cell Culture and Tissue Regeneration

To increase contact area for cell seeding and increasing mass transfer and interconnection of space, the present invention further provides a scaffold comprising 6 layers with hexagonal porous channel stacked in a spiral staircase way.

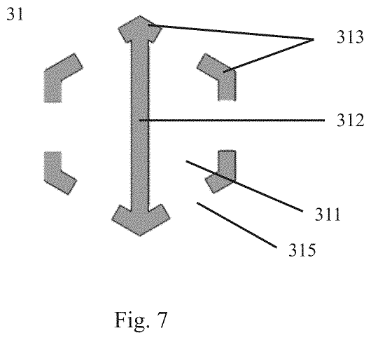

Referring to FIG. 7, the first layer 31 comprises a plurality of segments 313, and each of the first through holes 311 is framed by six of segments 313 indirectly connecting with one another. Preferably, the connecting bar 312 connects with two of segments 313. The plurality of segments 313 is arranged to form one of the first through holes 311, and the segments 313 are not connected with one another. In another aspect, a gap 315 is formed between two adjacent segments. Each of the segments 313 includes at least two portions intersected with each other for defining the first through holes 311.

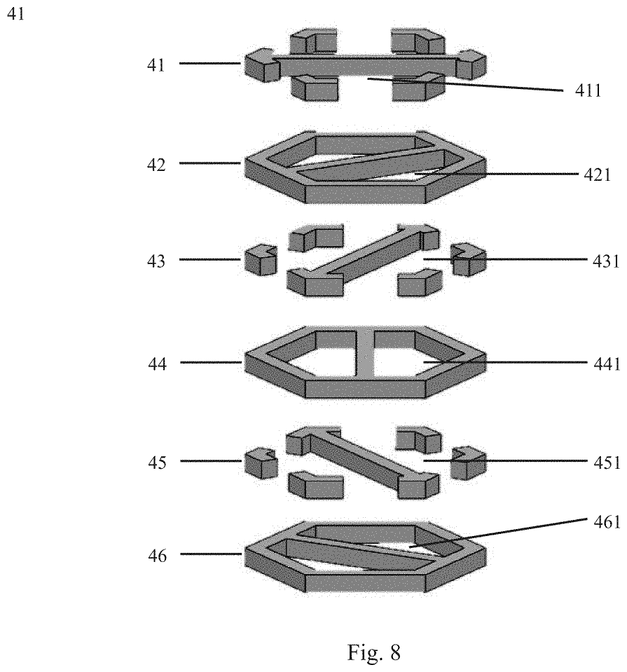

Referring to FIG. 8, the scaffold for cell culture and tissue regeneration comprises a first layer 41 comprising a plurality of first through holes 411; a second layer 42 comprising a plurality of second through holes 421, being adjacent to the first layer 41, a third layer 43 comprising a plurality of third through holes 431, a fourth layer 44 comprising a plurality of forth through holes 441, a fifth layer 45 comprising a plurality of fifth through holes 451, and a sixth layer 46 comprising a plurality of sixth through holes 461, wherein the third layer 43 is stacked on and adjacent to the fourth layer 44 in staggering order and the third layer 43 is also adjacent to and under the second layer 42 in staggering order, and the fifth layer 45 is stacked on and adjacent to the sixth layer 46 in staggering order and the fifth layer 45 is also adjacent to and under the fourth layer 44 in staggering order, wherein the third layer 43 and the fifth layer 45 are substantially same as the first layer 41 and the fourth layer 44 and the sixth layer 46 are substantially same as the second layer 42, and wherein one of the third through holes 431 is in communication with a corresponding one of the second through holes 421, in another embodiment, one of the first through holes 411 is in communication with a corresponding one of the second through holes 421, the third through holes 431, the fourth through holes 441, the fifth through holes 451 and the sixth through holes 461.

Preferably, configurations of the first through hole 411, the third through hole 431 and the fifth through holes are but different from the configurations of the second through hole 421, the fourth through holes 441 and the sixth through holes 461, which share the same configuration. The sides of the first through holes 411, the third through holes 431 and the fifth through holes 451 are discontinuous segments, and each of the first through holes 411, the third through holes 431 and the fifth through holes 451 is framed by six segments indirectly connecting with one another. On the other hand, the second through holes 421, the fourth through holes 441 and the sixth through holes have solid sides.

Referring to FIG. 8, the second through hole 421, the fourth through hole 441 and the sixth through hole 461 are enclosed through holes, and the first through hole 411, the third through hole 431 and the fifth through hole 451 are spaces surrounded by a plurality of discontinuous segments.

Preferably, the scaffold for cell culture and tissue regeneration is made of a biocompatible material; more preferably the biocompatible material is poly(glycerol sebacate) acrylate (PGSA). The physical properties of PGSA can be varied through different levels of acrylation modification of PGS. As PGSA is a photocurable and biodegradable polymer, high-resolution digital photoprocessor projector may be used to project a shape-specific light source onto the PGSA material to cure the layers, and the layers are stacked to form a three-dimensional structure for rapid and customized production of scaffolds with varible size.

### Example 3. The Evaluation of Differentiation of Embryonic Stem Cells (ESCs) on Different Substrates

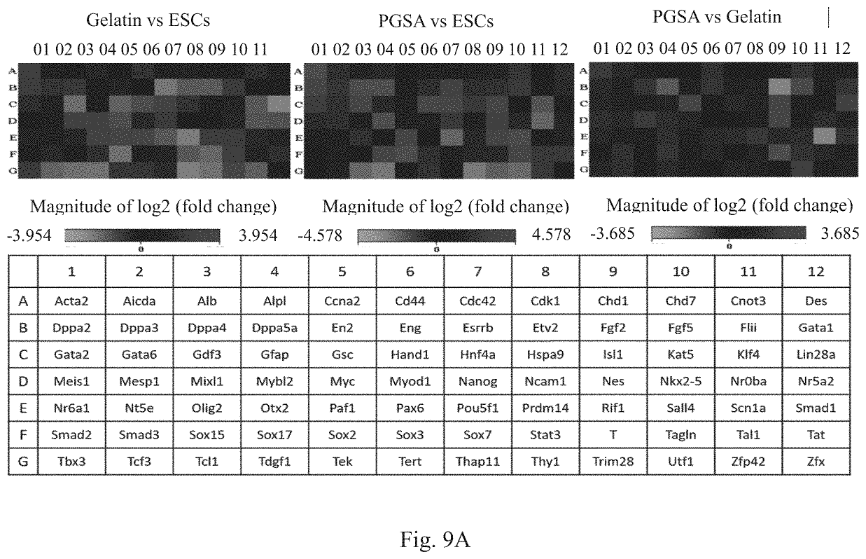

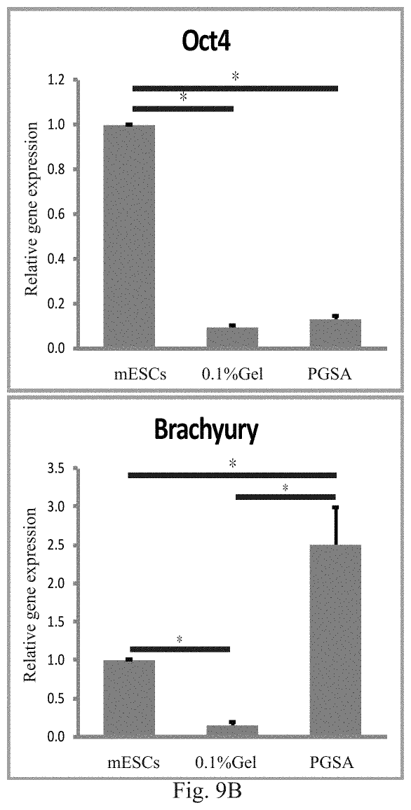

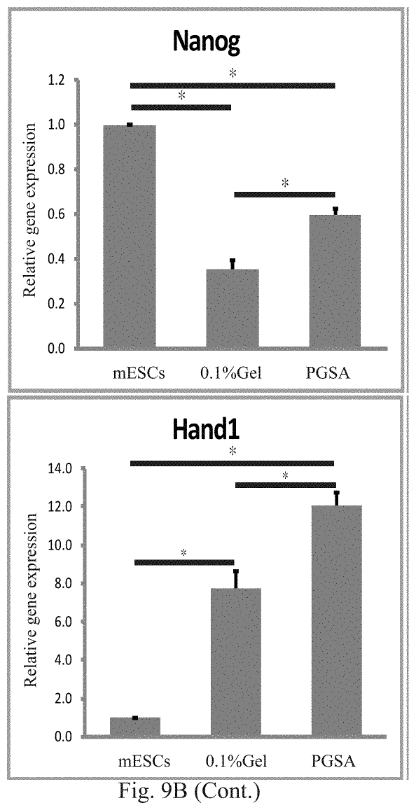

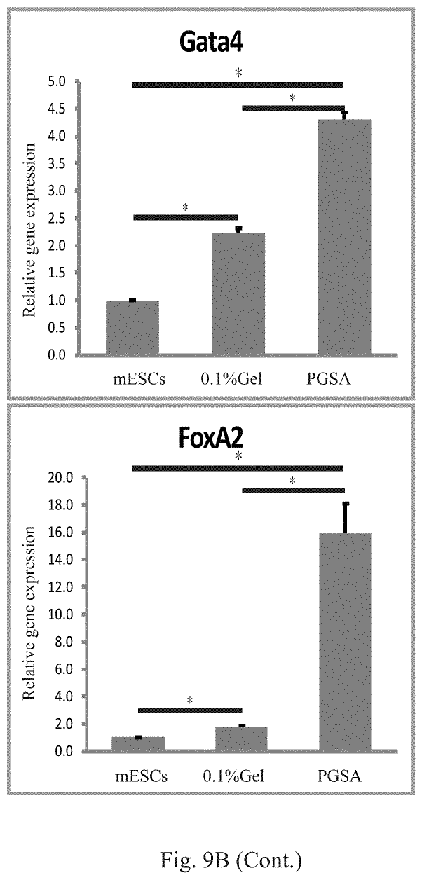

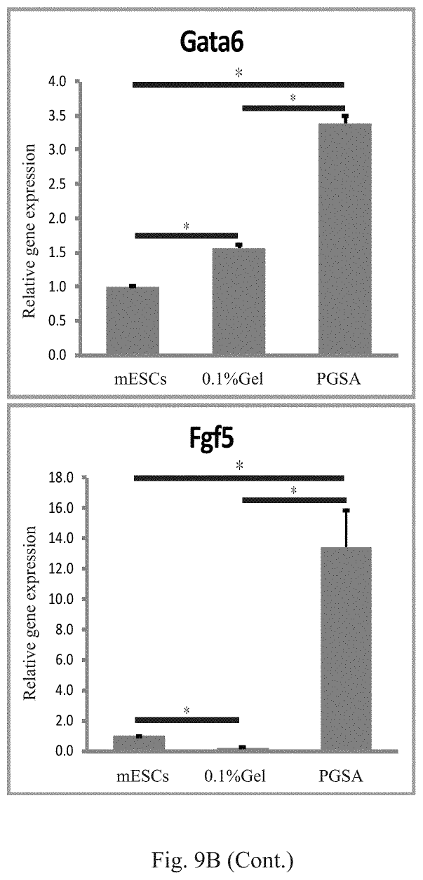

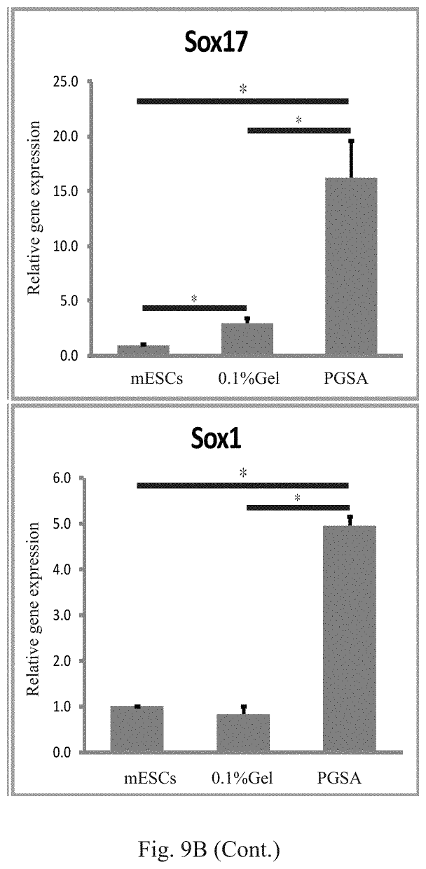

Given that extracellular substrate plays an important role in modulating stem cell fate, the effect of a novel photocurable and biocompatible polymeric material, PGSA, on stem cell differentiation is investigated in this example. PGSA with various materials on the differentiation of embryonic stem cells (ESCs) or vascular progenitor cells (VPCs) is compared. ESCs are plated on gelatin or PGSA, and induced spontaneous differentiation by withdrawing LIF in the culture medium. One week later, RT2 profiler PCR array analysis is performed to assess pluripotent and early differentiation marker gene expressions. Interestingly, endodermal markers (such as GATA6 and Sox17), mesodermal markers (such as brachyury and Mix11) and ectodermal marker FGF5 are highly upregulated in ESCs on PGSA compared to gelatin (FIG. 9A). To further verify this finding, quantitative Real Time-PCR (qRT-PCR) analysis of pluripotent and three-germ layer markers of ESCs cultured on PGSA or gelatin is performed. In comparison with ESCs on gelatin, ESCs on PGSA exhibit significantly reduced pluripotent markers Oct4 and Nanog whereas statistically elevated endodermal markers (GATA4, GATA6 and Sox17), mesodermal markers (brachyury, Hand1 and FoxA2) and ectodermal marker (FGF5 and Sox1) after spontaneous differentiation for one week (FIG. 9B).

### Example 4. Comparison of Vascular Lineage Differentiation of Vascular Progenitor Cells (VPCs) on Different Substrates

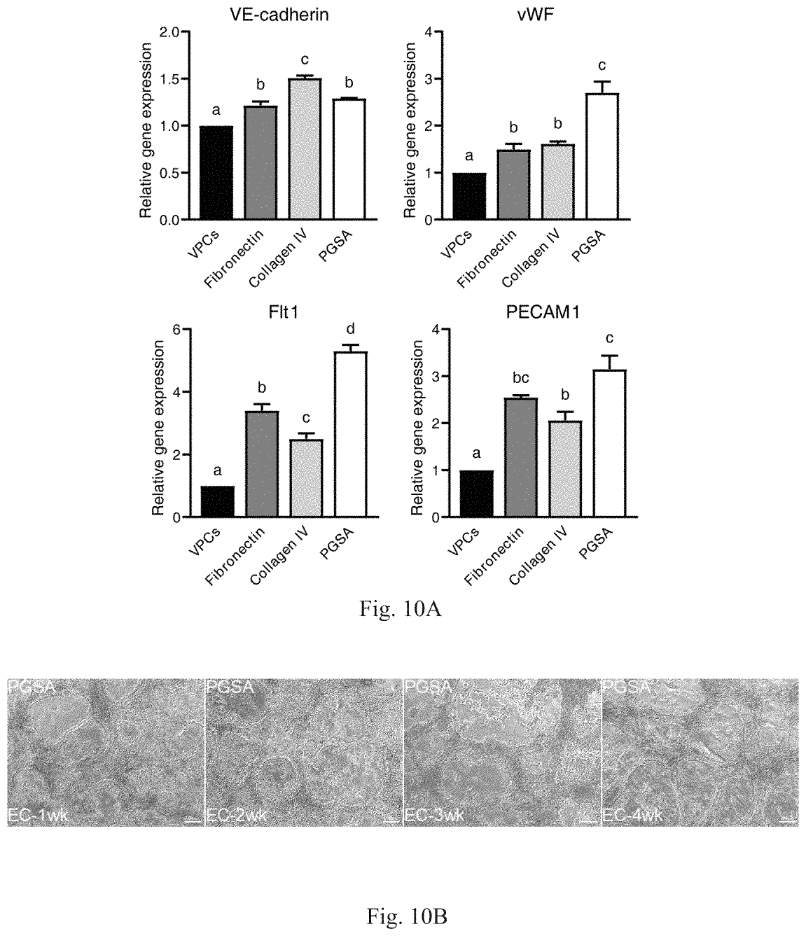

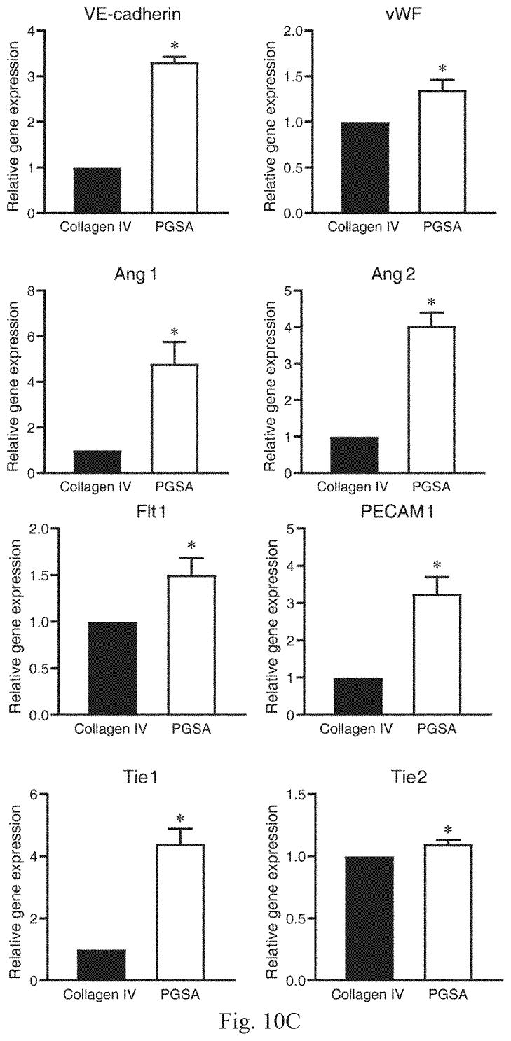

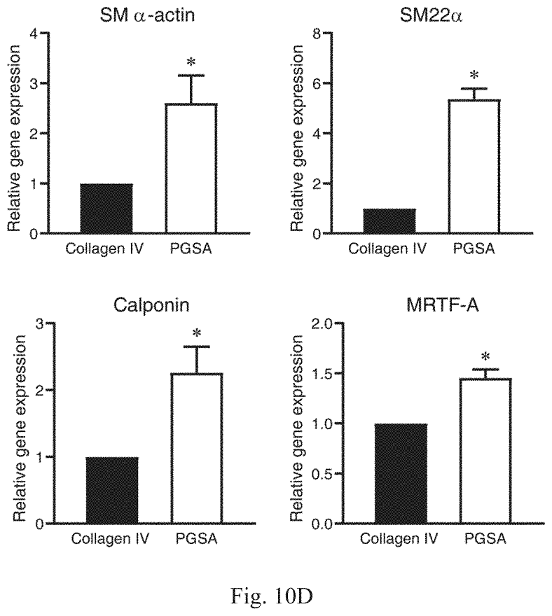

Vascular progenitor cells (VPCs) are intermediate cells during differentiation of ESCs to endothelial cells (ECs) and smooth muscle cells (SMCs). Previous studies have utilized fibronectin or collagen IV to provide an environment supporting the maintenance or differentiation of ECs. Thus qRT-PCR analysis is performed to assess EC development of VPCs on fibronectin, collagen IV and PGSA at the early one-week time point (EC-1 wk). All groups express higher levels of EC markers VE-cadherin, vWF, Flt1 and PECAM-1 than undifferentiated VPCs. Particularly, expressions of vWF, Flt1 and PECAM-1 in EC-1 wk on PGSA are significantly superior to fibronectin and collagen IV (FIG. 10A). Intriguingly, it is observed that VPCs have altered their morphology to tubular network, similar to that of vascular network, during four weeks EC induction on PGSA (FIG. 10B), suggesting increased angiogenic potential. Next, gene expressions are compared during long-term EC differentiation of VPCs on PGSA and collagen IV by qRT-PCR. Various EC development-associated genes, namely VE-cadherin, vWF, Flt1 and PECAM-1 are examined. It is found that gene expression levels of differentiating VPCs on PGSA after four weeks of EC induction are significantly higher than those on collagen IV (FIG. 10C). Similarly, differentiation of SMCs from VPCs on PGSA reveals that various SMC development-associated genes, such as SM α-actin, SM22α, calponin and MRTF-A are statistically higher than that on collagen IV (FIG. 10D). These results indicate that PGSA promotes mesodermal vascular cell differentiation from ESCs and VPCs.

### Example 5. The Preparation of Multiple 3D Structure of PGSA Scaffolds via Digital Light Processing-Additive Manufacturing System (DLP-AM)

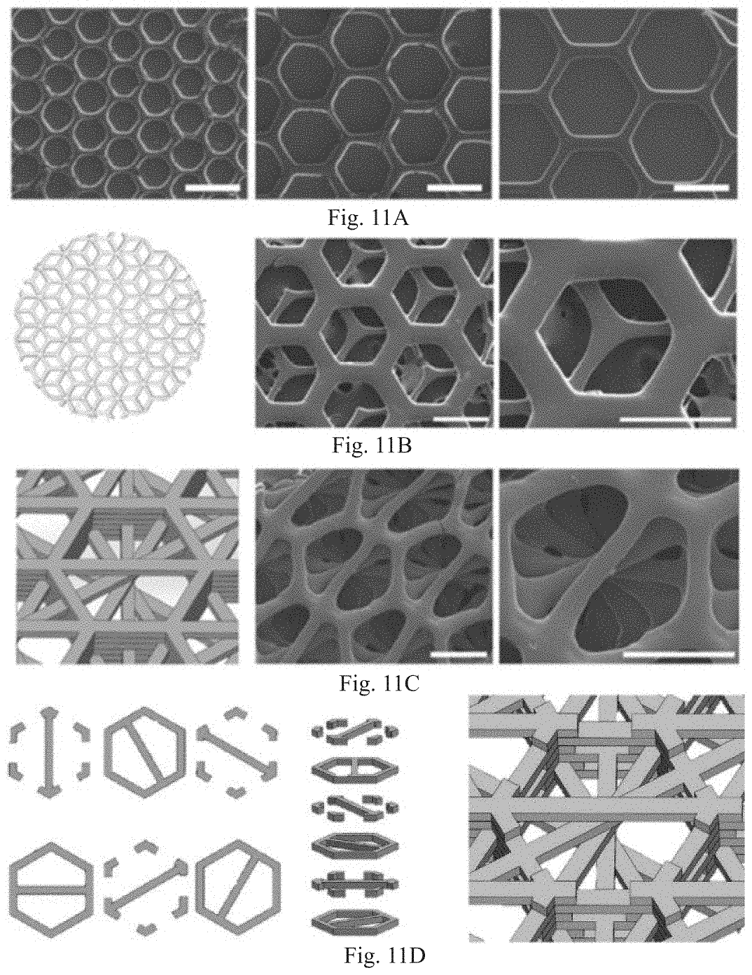

In order to facilitate regeneration of defective tissue, scaffolds with hexagonal shaped cavities are prepared via DLP-AM. Hexagonal wells are fabricated in three different sizes with the length of each edge at 173, 346 and 520 μm, and the height of the wells are uniformly 100 μm (FIG. 11A). With the clear increase in structure accuracy and integrity over increasing size of the hexagonal edges, it is therefore determined that when printing PGSA polymeric scaffolds hole the DLP-AM system, it is best printing structures 300 μm and above. Subsequently, hexagonal staggered holes are designed and printed. Two similar layers are stacked in a staggering order, similar to those of a honeycomb (FIG. 11B). To facilitate the cell seeding efficiency while maintaining medium circulation during cell culture, a hexagonal spiral-shaped structure is designed. Six layers of repeating hexagonal through-holes each with one rod in the center are stacked vertically, and the center rods are rotated by 30 degrees each layer counterclockwise. Hollow spaces that follow the outer rim of the rotating rods spiraling downward are also observed (FIG. 11C). Although the spiral staircase structure had increased the medium circulation vertically, it is considered that the designed through-holes might be filled up by cells and reduced the efficiency of medium circulation for longer culture. To this end, horizontal channels are also introduced to the staircase structures to facilitate the horizontal exchange of culture medium (FIG. 11D). Spiral staircase structure and horizontal porous channels help effectively increase mass transfer and interconnection of space.

Using the Said PGSA Scaffold to Generate a Pre-Endothelialized Engraftment Scaffold

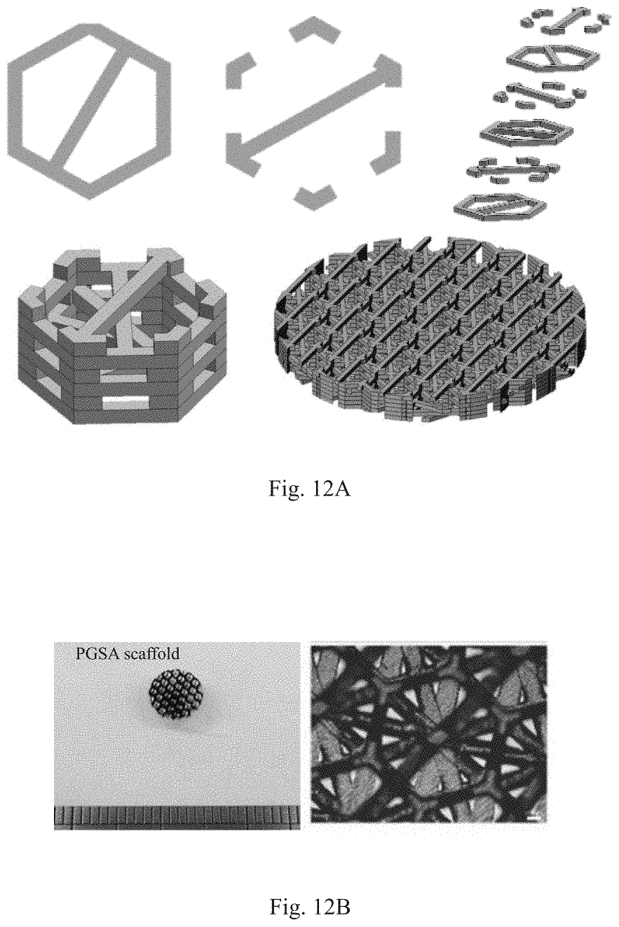

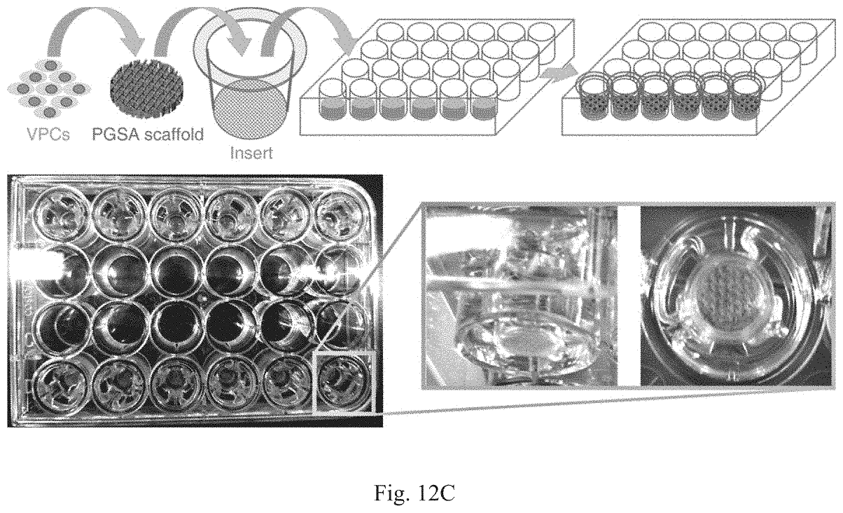

Regarding penetration of metabolites and nutrients in 3D scaffolds and efficacy of cellular adhesion, a novel hexagonal high diffusion staircase structure is designed by SolidWorks to produce 3D PGSA scaffolds (FIG. 12A). For proof of this concept, DLP-AM combined with PGSA is adopted to fabricate transplantable tissue scaffolds. After 3D printing, macro- and micro-structure of novel PGSA scaffolds are observed (FIG. 12B). In order for long-term culture, transwell suspension system is used for vascular constructs generated by populating novel PGSA scaffolds with VPC during EC differentiation (FIG. 12C). By combination of the cell-based and the scaffold-based strategies, assembly of differentiated endothelial cells and hexagonal staircase porous 3D-printed scaffold constitutes a high diffusion pre-endothelialized construct.

Comparison of Cell Seeding and Medium Diffusion Efficiency on Scaffolds with Different Structures

The effective surface area for the six designs is listed, which is directly correlated to the cell seeding efficiency on scaffolds. It is clear that in order to initiate higher cell seeding density while maintaining high medium diffusion to facilitate long-term cell culture, the flat, hexagonal staircase and high diffusion hexagonal staircase scaffolds are all good choices. However, it is clear that through the three-dimensional structures in the staircases, 3D growth in cells is triggered and thus is preferred for tissue engineering (Table. 1). Novel design of hexagonal staircase microstructure increases contact area of materials for cell seeding.

TABLE 1
Cell seeding and medium diffusion efficiency on different
structure of PGSA
3D-printed scaffold design factors

| | Effective | | | 3D |
|---|---|---|---|---|
| | Surface | Cell Seeding | Medium | Growth |
| Design | Area | Efficiency | Diffusion | of Cells |
| Flat | 100% | High | Medium | + |
| Hexagonal Well | 100% | High | Low | + |
| Hexagonal Staggered | 38.42% | Low | High | + |
| Hexagonal Sealed | 100% | Medium | Low | ++ |
| Staggered | | | | |
| Hexagonal Staircase | 73.73% | High | High | +++ |
| High Diffusion | 73.73% | High | High | +++ |
| Hexagonal Staircase | | | | |

* + biocompatible, ++ biocompatible and three dimensional cell growth, +++ biocompatible, three dimensional cell growth and long-term cell culture

In Vitro and In Vivo Test of Pre-Endothelialized Engraftment Scaffold

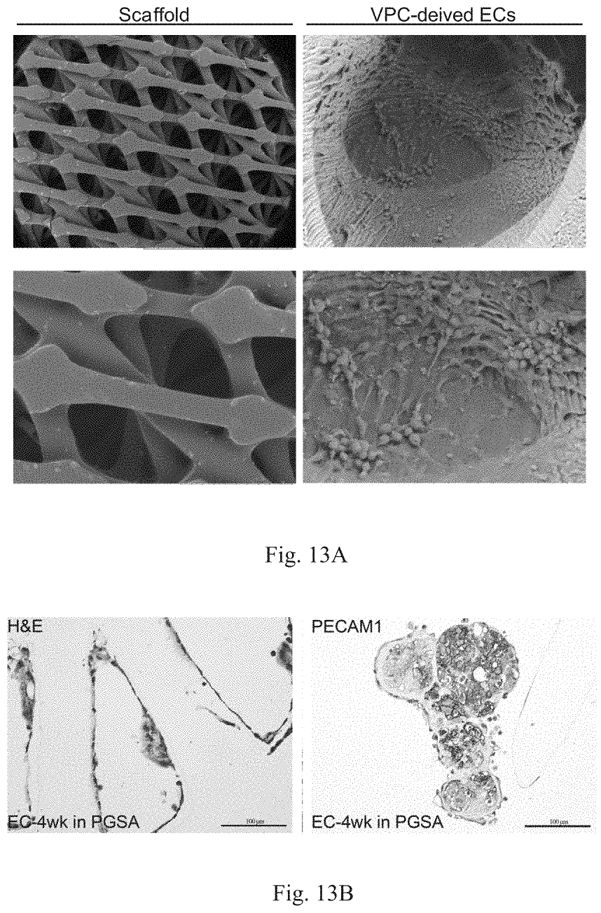

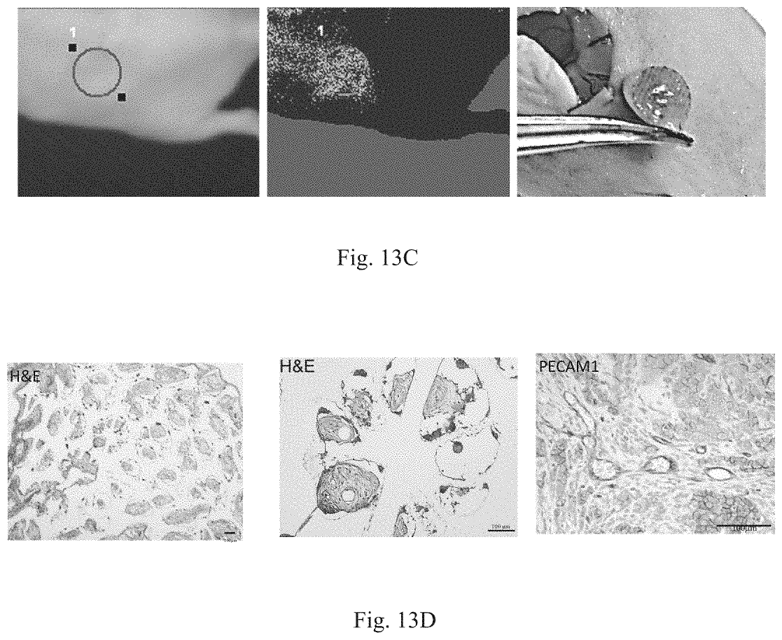

Scanning electron microscopy (SEM) images confirmed the complete high diffusion hexagonal staircase structure within PGSA scaffold. Cell seeding experiments indicated that this design is highly efficient for cell engraftment in 3D structures and facilitated mass transfer for long-term suspension culture (FIG. 13A). H&E staining presents the morphology of VPC-ECs on scaffold. Histochemical staining is carried out to further characterize the EC marker platelet and endothelial cell adhesion molecule 1 (PECAM1) expressed in vascular constructs post long-term suspension culture in vitro (FIG. 13B). After subcutaneous transplantation in Nude mice for four weeks, laser speckle contrast imaging indicates blood accumulation in transplanted site (FIG. 13 C). To further confirm this result, subcutaneous tissue of the transplanted site is harvested to validate. The appearance and histological analysis of the vascular constructs reveal the traces of scaffold structure and indeed engrafted into the region of subcutaneous tissue. In addition, it is found that the vascular constructs possess functional EC abilities whole exhibiting PECAM1 expressions in vivo (FIG. 13D). As the results, the high diffusion pre-endothelialized construct enhances the vascularization in vivo.

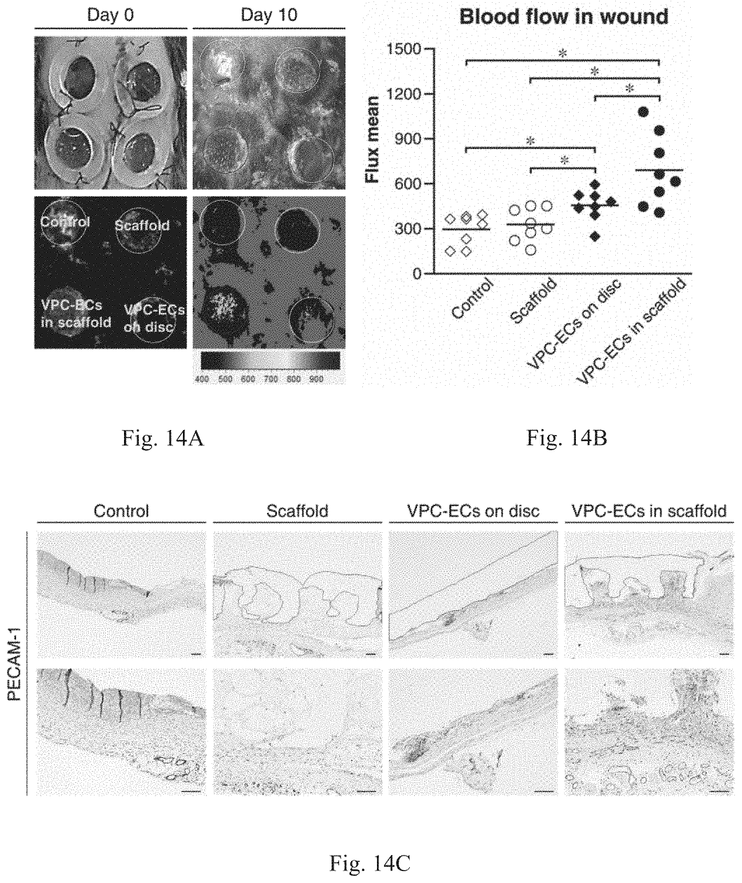

To further validate the functions of vascular constructs, symmetrical wound healing mouse model is utilized for transplantation. After creating 4 wounds in a mouse, wounds are treated with PBS as control, or transplanted with scaffold only, disc with VPC-ECs, or 3D scaffold with VPC-ECs for 10 days. By laser speckle contrast imaging analysis, higher intensity of blood flow is found in the site after implantation of VPC-ECs on scaffold (FIG. 14A). When quantitatively measured, the blood flux mean in VPC-ECs on scaffold is significantly superior to the others (FIG. 14B). After implantation of vascular constructs for 10 days, the transplanted site is harvested for validation. Histochemical staining reveals that the VPC-ECs on scaffold indeed engrafted into the region of lesion. In addition, it is found that the VPC-ECs on scaffold possess functional angiogenesis ability, as well as high levels of PECAM1 (FIG. 14C). The high diffusion pre-endothelialized construct can be applied as a blood vessel system for engineered tissues and organs in vitro.

In the present invention, 3D printing approach is utilized to develop an innovative 3D vascular architecture that provides an optimum spatial structure for oxygen and nutrient diffusion.

The results as described above demonstrate that PGSA-based 3D printing offer a promising technology for vascular tissue engineering. The novel customized scaffold rapidly fabricated via 3D-printing using biocompatible and biodegradable elastomer has hexagonal rotating staircase with high surface area and high culture medium diffusivity. Such scaffolds combined with cells are especially promising for tissue models (research use) and applications of therapies (clinical use) in the future. Collectively, the scaffold of this invention is applicable to multiple tissue engineering disciplines.

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

1. A scaffold with staircase microstructure for cell or tissue culture, comprising:
a first layer comprising a plurality of first through holes with a regular polygon shape; and
a second layer comprising a plurality of second through holes with a regular polygon shape;
wherein the first layer is stacked on and adjacent to the second layer in staggering order, and one of the first through holes is in communication with a corresponding one of the second through holes; and wherein the center of each first through hole is respectively aligning with a vertex of the corresponding second through hole.

2. The scaffold according to claim 1, wherein at least two of the second through holes are in communication with one of the first through holes.

3. The scaffold according to claim 1, wherein a size of the first through holes is substantially same as a size of the second through holes.

4. The scaffold according to claim 1, wherein an orientation of the first through holes differs from an orientation of the second through holes.

5. The scaffold according to claim 1, wherein the first through holes and the second through holes are enclosed through holes.

6. The scaffold according to claim 1, wherein each of the first through holes and the second through holes further comprising a connecting bar disposed.

7. The scaffold according to claim 6, wherein the connecting bar is disposed within one of the first through holes and one of the second through holes, and the scaffold for cell culture further comprises a central bar connecting the connecting bar.

8. The scaffold according to claim 1, wherein the sides of the first through holes are discontinuous segments, and each of the first through holes is framed by six segments indirectly connecting with one another.

9. The scaffold according to claim 8, wherein each of the connecting bars of the first layer connects with two segments.

10. The scaffold according to claim 8, wherein a gap is formed between two adjacent segments.

11. The scaffold according to claim 8, wherein each of the segments includes at least two portions intersected with each other.

12. The scaffold according to claim 1, wherein the scaffold is made of poly(glycerol sebacate) acrylate (PGSA).

13. The scaffold according to claim 8, wherein each of the second through holes is an enclosed through hole, and each of the first through holes is a space surrounded by a plurality of discontinuous segments.

14. The scaffold according to claim 1, wherein the scaffold further comprises multiple copies of the first layer and the second layer.

15. The scaffold according to claim 14, wherein the multiple copies are two copies of the first layer and the second layer, which are a third layer with a plurality of third through holes, a fourth layer with a plurality of fourth through holes, a fifth layer with a plurality of fifth through holes, and a sixth layer with a plurality of fifth through holes, respectively.

16. The scaffold according to claim 15, wherein the third layer and the fifth layer are substantially as same as the first layer, and the fourth and the sixth layer are substantially as same as the second layer.

17. The scaffold according to claim 15, wherein the third layer is stacked on and adjacent to the fourth layer in staggering order and the third layer is also adjacent to and under the second layer in staggering order, and the fifth layer is stacked on and adjacent to the sixth layer in staggering order and the fifth layer is also adjacent to and under the fourth layer in staggering order to form a scaffold with six layers stacked in a spiral staircase way.

18. The scaffold according to claim 17, wherein each of the second through holes, the fourth through holes and the sixth through holes is an enclosed through hole, and each of the firth through hole, third through holes and the fifth through holes is a space surrounded by a plurality of discontinuous segments.

19. A method for culturing a vascularization tissue comprising culturing a cell with the scaffold according to claim 1.

20. The method according to claim 19, wherein the cell is an embryonic stem cell or vascular progenitor cell.

21. A method for enhancing differentiation of a stem cell comprising culturing the stem cell with the scaffold according to claim 1.

22. The method according to claim 21, wherein the stem cell is an embryonic stem cell.

23. A method for enhancing differentiation of a vascular cell comprising culturing the vascular cell with the scaffold according to claim 1.

24. The method according to claim 21, wherein the vascular cell is a vascular progenitor cell.

25. A method for enhancing vascularization in a wound comprising:
culturing a vascular cell with the scaffold according to claim 1 for generating a pre-endothelialized engraftment scaffold, and
engrafting the pre-endothelialized engraftment scaffold into the wound.

26. The method according to claim 25, wherein the vascular cell is a vascular progenitor cell.
